(19) United States
(12) Patent Application Publication
(10) Pub. No.: US 2023/0321289 A1
(43) Pub. Date: Oct. 12, 2023

(54) METHOD FOR STERILIZING AND DECONTAMINATING POST-CONSUMER ABSORBENT SANITARY PRODUCTS CONTAMINATED BY ORGANIC COMPOUNDS DERIVING FROM HUMAN METABOLISM

(71) Applicant: The Procter & Gamble Company, Cincinnati, OH (US)

(72) Inventors: Giorgio VACCARO, Spoltore (IT); Fabio PAGOTTO, Spoltore (IT); Tonino CARUSO, Spoltore (IT)

(21) Appl. No.: 18/209,644

(22) Filed: Jun. 14, 2023

Related U.S. Application Data
(63) Continuation of application No. PCT/IB2021/061635, filed on Dec. 13, 2021.

(30) Foreign Application Priority Data
Dec. 21, 2020 (IT) 102020000031721

(51) Int. Cl. A61L 2/06

(52) U.S. Cl. CPC A61L 2/06; A61L 2202/26

(57) ABSTRACT

A method for sterilizing and decontaminating post-consumer absorbent sanitary products contaminated by organic compounds deriving from human metabolism and comprising drug residue, said post-consumer absorbent sanitary products comprising fractions of plastic, super-absorbent polymers (SAPs), and optionally cellulose, the method comprising at least the steps of: i) sterilizing (SR) said post-consumer absorbent sanitary products by heating to a temperature equal to or lower than 140° C. and at a pressure comprised between 1 bar and 3.6 bar to obtain sterilized post-consumer absorbent sanitary products; and ii) decontaminating (DC) from organic compounds said sterilized post-consumer absorbent sanitary products by means of an oxidizing treatment. The oxidizing treatment is carried out by putting the sterilized post-consumer absorbent sanitary products in contact with a gas containing ozone preferably at a temperature equal to or higher than 60° C., more preferably comprised between 60° C. and 80° C. The sterilized post-consumer absorbent sanitary products subjected to the decontaminating step have a humidity of less than 80%, preferably comprised between 60% and 75%.

### FIELD OF THE INVENTION

The present disclosure relates to recycling of post-consumer absorbent sanitary products. In particular, the present disclosure regards methods for sterilizing and decontaminating post-consumer absorbent sanitary products for humans from organic compounds deriving from human metabolism.

### BACKGROUND OF THE INVENTION

Absorbent sanitary products for humans are generally made up of various materials, amongst which, for example, films of plastic material, cellulose fluff, superabsorbent polymers (SAPS), and transpirant sheets made of synthetic fibrous material. Such sanitary products consequently contain high-quality materials, recovery of which for re-use on the market is a decidedly desirable aim. Critical aspects linked to the treatment of post-consumer absorbent sanitary products regard not only the presence of organic excretions and bacterial contamination but also the presence of chemical compounds of a post-metabolic nature that derive from drugs used by the user for specific therapeutic treatments.

Post-consumer absorbent sanitary products must consequently be not only sterilized but also decontaminated from a chemical standpoint to be subsequently recycled and marketed as recycled raw materials (and not as refuse).

Exposure of post-consumer absorbent sanitary products to a sterilization temperature, however, may not be sufficient to obtain also decontamination from organic residue of a post-metabolic nature, for example deriving from drugs.

On the other hand, methods that envisage subjecting post-consumer absorbent sanitary products to heating steps at very high temperature and pressure may present non-negligible critical aspects. Methods are today known, for example described in the document EP 3 162 455 B1, which envisage treatment of post-consumer absorbent sanitary products at a temperature of at least 200° C. and at a pressure higher than 20 bar. Such temperature and pressure regimes, however, albeit potentially effective also from a standpoint of decontamination from chemical residue of a post-metabolic nature, may prove decidedly aggressive for the mixed material subjected to the treatment. in particular, the cellulose-based component (a carbohydrate consisting of glucose units) undergoes browning beyond 140° C., caramelization beyond 160° C., and de-polymerization beyond 200° C., with consequent reduction of the softness and absorption capacity, whereas plastic starts to soften beyond 160° C., until it melts englobing the other materials, and consequently losing its intrinsic mechanical properties. Consequently, the yield and quality of the recycled material may be jeopardized.

As an alternative, absorbent sanitary products may be treated with chemical oxidants, such as hydrogen peroxide, persulphate, peroxymonosulphate, and ozone.

As described, for example, in the documents WO 2019/084203 A1, RO 129 948 B1, and US 2011/076192 A1, the oxidative action of such compounds has been used in methods for treating surgical instruments, medical devices, electronic devices, surfaces (e.g., work spaces, patient rooms, and organic material).

In the case of post-consumer absorbent sanitary products to be treated for convenient recycling, however, the oxidative action of the aforesaid compounds—which must not he limited to just chemical decontamination, but also to a bactericidal, bleaching, and deodorant action—may entail de-structuring of the SAPs and depolymerization of the cellulose fibres.

Furthermore, even though oxidants such as hydrogen peroxide and ozone do not release particular contamination as they decompose into water and oxygen respectively, they may cause—in given operating conditions—formation of peroxides, which are particularly reactive species that may form in situ and trigger combustion of the solid organic material present.

The document KR 101 044 439 B1, for example, describes methods for treating used diapers by means of exposure thereof to washing and sterilizing steps, where ozone is introduced into the sterilization reactor simultaneously with exposure of the post-consumer absorbent sanitary products to the flow of water.

The ozone dissolved in water, however, presents a reactivity lower than that of gaseous ozone and moreover the high degree of humidity of the material exposed to the flow of water implies the need i) to squeeze the material following upon the sterilizing step and ii) to open up the material in order to expose it to the subsequent drying step. Exposure of the material to washing steps and the flow of water moreover entails the need to envisage treatment and disposal of waste water.

### OBJECT AND SUMMARY OF THE INVENTION

The object of the present description is to overcome the aforesaid drawbacks and to provide a method for recycling post-consumer absorbent sanitary products that will enable sterilization and decontamination from organic compounds deriving from human metabolism, for example drug residue, at the same time preserving the quality of the products recovered from the post-consumer material (plastic, super-absorbent polymers—SAPs, and optionally cellulose) for convenient re-use or recycling on the market.

Post-consumer absorbent sanitary products to be subjected to the method of the present disclosure may, for example, comprise diapers for babies, incontinence pads for adults, sanitary napkins for women, and bed linings. These absorbent sanitary products may comprise plastic, super-absorbent polymers, cellulose, or else even just plastic and super-absorbent polymers.

According to the present description, the above object is achieved thanks to a method having the characteristics forming the subject of the annexed claims. The claims form an integral part of the teaching provided herein in relation to the method described.

An embodiment of the present disclosure provides a method for sterilizing and decontaminating post-consumer absorbent sanitary products contaminated by organic compounds deriving from human metabolism and comprising drug residue, said post-consumer absorbent sanitary products comprising fractions of plastic, super-absorbent polymers (SAPS), and optionally cellulose, the method comprising the steps of:
sterilizing SR said post-consumer absorbent sanitary products by heating to a temperature equal to or lower than 140° C. and at a pressure comprised between 1 bar and 3.6 bar to obtain sterilized post-consumer absorbent sanitary products; and
decontaminating DC said sterilized post-consumer absorbent sanitary products from said organic compounds by means of an oxidizing treatment, wherein said oxidizing treatment is carried out by setting in contact the sterilized post-consumer absorbent sanitary products with a gas containing ozone at a temperature equal to or higher than 60° C., preferably comprised between 60° C. and 80° C.

The sterilized post-consumer absorbent sanitary products subjected to the treatment with ozone have a humidity of less than 80%, preferably comprised between 60% and 75%.

The humidity of the sample is the percentage of water contained therein and is calculated by subtracting from 100 the content of dry substance obtained by drying a known amount of sample up to constant weight (IRSA-CNR Procedures 1-10-manuale_3_2011_compost-2: Umidita e sostanza secca).

Treatment of post-consumer absorbent sanitary products with gaseous ozone, maintaining the humidity thereof at a value of lower than 80%—hence not exposing them to steps of washing and immersion in water—presents a series of advantages over oxidative treatments that contemplate the use of water. First of all, gaseous ozone has proven more reactive than ozone dissolved in water, and the plant for oxidative treatment is simpler and hence economically more advantageous. The treated material then does not have to be subjected to squeezing steps and is conveyed directly into a drier. The method moreover does not generate waste water and sewage that has to be treated and then disposed of

The method forming the subject of the present disclosure may further comprise the step of shredding SH the sterilized and decontaminated post-consumer absorbent sanitary products to obtain shredded post-consumer absorbent sanitary products having a particle size of less than 10 cm, preferably less than 3 cm, even more preferably less than 1 cm.

The method may further comprise the step of drying DR the shredded post-consumer absorbent sanitary products to obtain shredded and dried post-consumer absorbent sanitary products comprising plastic, SAP, and optionally cellulose.

Moreover, the method may comprise the step of separating plastic, cellulose, and SAP from said shredded and dried post-consumer absorbent sanitary products.

In one or more embodiments, the decontaminating step DC may be carried out following the sterilizing step SR and prior to the shredding step SH.

The method forming the subject of the present disclosure favours obtaining the separate sterilized components of cellulose, plastic, and SAP, decontaminated by organic compounds, such as residue of a post-metabolic nature deriving from drugs. These components present bleached and without unpleasant odours. These features render them suited to convenient re-use.

The subject of the present disclosure is moreover plastic, super-absorbent polymers (SAP), and cellulose obtained with the method described that have been separated from post-consumer absorbent sanitary products decontaminated by organic compounds deriving from human metabolism and comprising drug residue.

### BRIEF DESCRIPTION OF THE DRAWINGS

The method will now be described in detail with reference to the attached drawings, which are provided purely by way of non-limiting example and in which:

FIG. 1 illustrates a diagram of a method known in the art for sterilizing and separating plastic, super-absorbent polymers (SAP), and cellulose from post-consumer absorbent sanitary products;

FIG. 2 is a top plan view of an apparatus that can be used for the method represented schematically in FIG. 1;

FIG. 3 illustrates a diagram of a method according to an embodiment of the present description envisaged in which is a step of decontamination front chemical compounds carried out downstream of the sterilizing step and prior to the shredding step; and

FIG. 4 illustrates a diagram regarding production and introduction of gaseous ozone into a reactor according to one embodiment of the present description.

### DETAILED DESCRIPTION

In the ensuing description, numerous specific details are provided to enable an in-depth understanding of embodiments. The embodiments may be implemented without one or more of the specific details or with other methods, components, materials, etc. In other cases, well-known structures, materials, or operations are not illustrated or described in detail so that aspects of the embodiments will not be obscured.

In all of the attached figures, unless otherwise specified in the context, parts or elements that are similar are designated by the same references and numerals, and the corresponding description will not be repeated for brevity.

Throughout the present description reference to “one embodiment” or “an embodiment” indicates that a particular aspect, structure or characteristic described with reference to the embodiment is included in at least one embodiment. Thus, the phrases such as “in one embodiment” or “in an embodiment” or the like that may be present in various points of the description do not necessarily all refer to one and the same embodiment. Moreover, the particular aspects, structures, or characteristics may be combined in any suitable way in one or more embodiments. The section headings used herein are provided merely for convenience and do not interpret the scope or purpose of the embodiments.

As anticipated in the foregoing sections, methods for sterilizing post-consumer absorbent sanitary products may not guarantee decontamination from organic residue of a post-metabolic nature, for example residue deriving from drugs.

The present Inventors have identified specific operating conditions of a method capable of favouring sterilization and at the same time decontamination from organic compounds of post-consumer absorbent sanitary products without any need to resort to operating conditions (for example, steps of heating to a temperature higher than 200° C. and at a pressure higher than 20 bar) that could vitiate the quality of the components separated and recovered from such post-consumer absorbent sanitary products, such as cellulose, plastic, and super-absorbent polymers (SAP).

In particular, the method forming the subject of the present disclosure is a method for sterilizing and decontaminating post-consumer absorbent sanitary products contaminated by organic compounds deriving from human metabolism and comprising drug residue, said post-consumer absorbent sanitary products comprising fractions of plastic, super-absorbent polymers (SAP), and optionally cellulose, the method comprising the steps of:
sterilizing SR said post-consumer absorbent sanitary products by heating to a temperature equal to or lower than 140° C. and at a pressure comprised between 1 bar and 3.6 bar to obtain sterilized post-consumer absorbent sanitary products; and
decontaminating DC the sterilized post-consumer absorbent sanitary products from said organic compounds by means of an oxidizing treatment;
wherein said oxidizing treatment is carried out by setting the sterilized post-consumer absorbent sanitary products in contact with a gas containing ozone; and wherein said oxidizing treatment is carried out at a temperature equal to or higher than 60° C., and preferably comprised between 60° C. and 80° C.

The sterilized post-consumer absorbent sanitary products subjected to the decontaminating step have a content of humidity of less than 80%, preferably comprised between 60% and 75%.

The humidity of the sample indicates the amount of water contained therein (expressed as percentage amount) and is calculated by subtracting from 100 the content of dry substance obtained by drying a known amount of sample until constant weight is reached. Drying of the sample, previously weighed, °, is generally performed in a stove, at a temperature comprised between 103° C. and 105° C. and for a period of at least 4 h, normally at constant weight. At the end of drying in the stove, the sample is again weighed, and the content of dry substance, DS, is obtained by applying the following formula: DS=P/P° 100, where is the weight in grams of the sample after dehydration, and P° is the weight in grams of the sample prior to dehydration. The humidity of the sample is calculated by subtracting from 100 the content in dry substance DS (IRSA-CNR Procedures 1-10-manuale_3_2011_compost-2: Umidita e sostanza secca: procedures IRSA-CNR 1984 and UNICHIM 10780/1998).

Advantageously, the post-consumer absorbent sanitary products subjected to the various steps of the method are not set in contact with aqueous solutions, nor are they immersed in aqueous solutions. The method does not envisage steps of washing in water, with consequent formation of sludge to be treated and then disposed of.

Designated by the expression “absorbent sanitary products” are in general disposable absorbent sanitary products, for example diapers for babies, incontinence pads for adults, sanitary napkins for women, and bed linings The absorbent sanitary products may comprise plastic, SAP, cellulose or else even just plastic and SAP.

In one or more embodiments, the sterilizing step may be carried out by heating the post-consumer absorbent sanitary products to a temperature comprised between 120° C. and 140° C., The time interval during which the sterilizing step is carried out may range between 20 min and 2 h.

The sterilizing step, like the decontaminating step, may be carried out in a closed reactor, preferably in an autoclave, more preferably a rotary autoclave that enables the post-consumer sanitary products to be kept moving.

In one or more embodiments, the method comprises a step of moving the post-consumer absorbent sanitary products subjected to the sterilizing step and the decontaminating step.

The oxidizing treatment of the decontaminating step envisages, preferably consists in, setting the sterilized post-consumer absorbent sanitary products in contact with a gas containing ozone. This treatment does not comprise steps of immersion of the post-consumer absorbent sanitary products in water or aqueous solutions. The decontaminating step by means of oxidizing treatment does not comprise the use of further oxidants other than ozone.

Keeping the humidity of the absorbent sanitary products at a value of lower than 80%—thus preventing their exposure to steps of washing and immersions in water—favours a greater reactivity of the ozone, avoids the need to subject the products to squeezing steps, and does not generate waste water and sludge that has to be treated and then disposed of.

The oxidizing treatment with the ozone-containing gas is carried out at a temperature equal to or higher than 60° C. preferably comprised between 60° C. and 80° C.

The concentration of ozone in the gas may be comprised between 0.06 kg/m³ and 0.14 kg/m³; preferably it is equal to 0.08 kg/m³. The oxidizing treatment with gaseous ozone may be carried out at a pressure comprised between 0.3 bar and I bar, preferably equal to 0.5 bar,

Preferably, the oxidizing treatment may be carried out for a period of time comprised between 30 min and 90 min, preferably for a period of 60 min.

Thanks to the specific operating conditions, the method forming the subject of the present disclosure enables products recovered front the post-consumer material to be obtained—cellulose, plastic, SAP—where the quality is preserved so that it can be conveniently marketed for re-use.

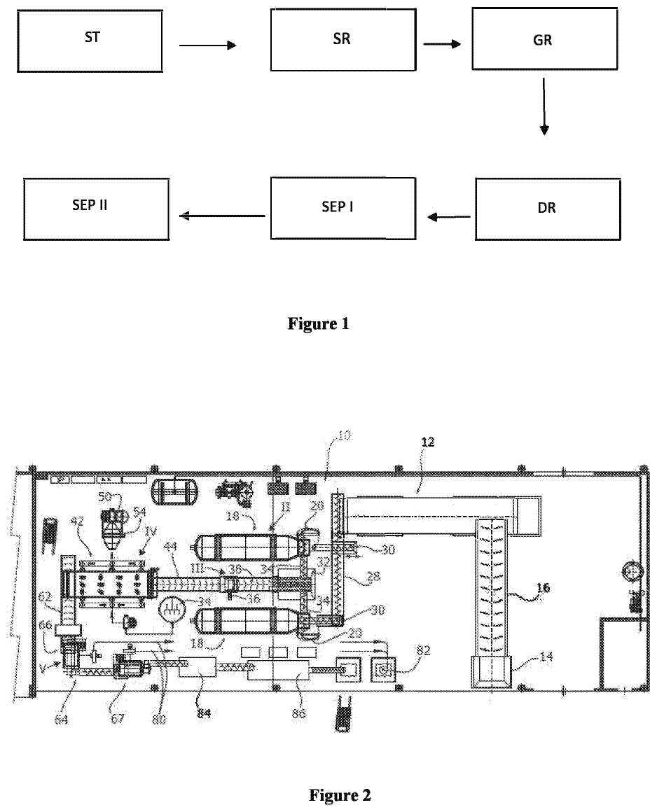

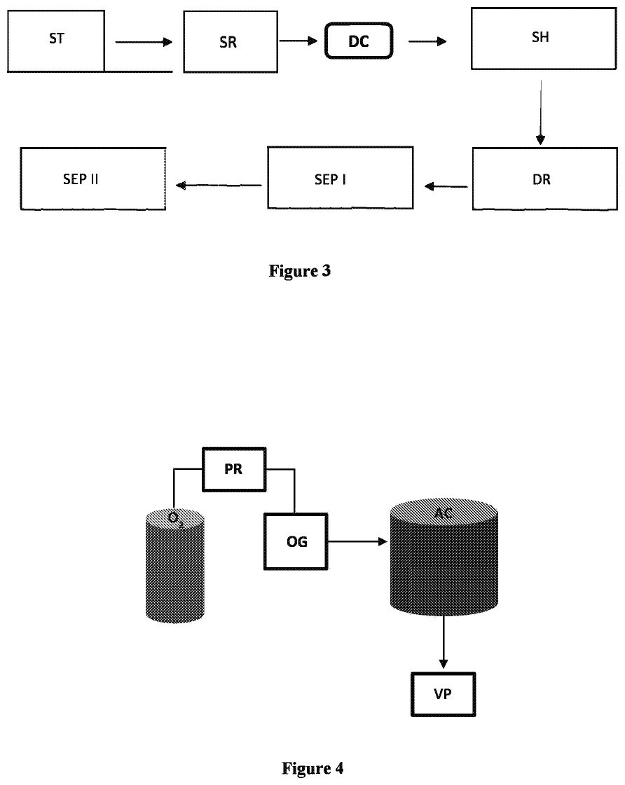

As schematically illustrated, for example in FIG. 1 and FIG. 3, the method may further comprise the steps of shredding the post-consumer absorbent sanitary products SH, drying the shredded products DR, separating SEP I the shredded and dried products into plastic and cellulose, and separating SEP II the cellulose into SAP and cellulose fluff, as for example described in the document WO 2018/060827 filed in the name of the present applicant.

In particular, the method forming the subject of the present disclosure may comprise a step of collecting ST the post-consumer absorbent sanitary products coming from the differentiated collection into an accumulation container. FIG. 2 illustrates an apparatus 10 in which the accumulation container is designated by reference number 12. The refuse-collection vehicles unload the post-consumer absorbent sanitary products in a dumping area 14, and a conveyor 16 loads the post-consumer absorbent sanitary products into the accumulation container 12. The post-consumer absorbent sanitary products collected may present a density in the region of 150-300 kg/m³ and a humidity in the region of 65-80%.

The humidity of the material, understood as percentage amount of water contained therein, is calculated from the dry weight of the sample (IRSA-CNR Procedures 1-10-manuale_3_2011_compost-2: Umidita e sostanza secca: procedures IRSA-CNR 1984 and UNICHIM 10780/1998).

The collection step ST is followed by the sterilizing step SR, for example carried out by loading the products into a rotary autoclave 18. In the example illustrated in FIG. 2, the apparatus 10 comprises two autoclaves 18 that are loaded alternately with post-consumer absorbent sanitary products coming from the accumulation container 12. A conveyor 28 picks up the products from the accumulation container 12 and conveys them to the autoclaves 18. Two loaders 30 load the products into the respective autoclaves 18. During loading of the products, the hatches 20 of the autoclaves are open, and the cylindrical body is driven in rotation to move the products progressively to the rear part. Once loading is through, the hatch 20 is closed and the autoclave 18 is heated and pressurized by means of direct and indirect supply of steam, until a temperature of approximately 135° C. and an internal pressure of approximately 3.1 bar is reached. During the sterilization treatment, the autoclave can be driven in rotation in a clockwise direction and a counterclockwise direction alternately about to its own axis in order to enable movement of the products contained therein. The sterilizing step SR has the purpose of bringing the temperature of the products up to above 121° C., i.e., to a temperature at which it is possible to obtain complete sterilization of the bacterial load. The sterilizing step may be carried out for a time interval comprised between 20 min and 2 h.

At the end of the sterilization treatment, the steam contained within the autoclave 18 is extracted and depurated in a scrubber 34. Then the hatch 20 is opened, and the body is driven in rotation to unload the products. In the example of FIG. 2, two autoclaves 18 are provided that operate in alternation. While a first autoclave 18 is performing the sterilization treatment, the other autoclave 18 performs the operations of unloading of the sterilized material and of loading of a new batch. In this way, downstream of the autoclaves 18 it is possible to obtain a substantially continuous flow of sterilized material. The sterilized material may have a density of approximately 300-400 kg/m³, a temperature of 80-100° C., and a humidity of around 70-85%.

The method forming the subject of the present disclosure, as schematically represented in FIG. 3, envisages carrying out a step of decontamination DC of the sterilized post-consumer absorbent sanitary products from chemical compounds deriving, for example, from drugs downstream of the sterilizing step SR. In particular, the oxidizing treatment using a gas containing ozone is carried out on the loads of post-consumer absorbent sanitary products already subjected to sterilization and contained in the autoclave.

The present Inventors have noted that the effectiveness of the decontamination increases when a vacuum condition is created in the reactor prior to introduction of the ozone-containing gas. The vacuum condition is obtained, for example, using a vacuum pump connected to the reactor, as represented schematically in FIG. 4. The vacuum pump VP generates in the reactor a relative pressure that may be comprised between −0.6 bar and −0.8 bar. Creation of the vacuum in the reactor prior to introduction of the ozone-containing gas enables reduction of the content of air and optimization of the content of gaseous ozone.

After the vacuum has been created, the gas containing gaseous ozone is introduced into the autoclave AC, in contact with the sterilized products, which, at this stage, have a humidity of less than 80%, preferably comprised between 60% and 75%.

Treatment with the ozone-containing gas is carried out keeping the temperature in the autoclave equal to or higher than 60° C., preferably comprised between 60° C. and 80° C.

The ozone-containing gas may be produced, for example using an ozone generator. As schematically illustrated in FIG. 4, for production of gaseous ozone liquid oxygen (O2) may be used, contained in a tank at the pressure of 200 bar, for example supplied by specialized firms that transport it with tankers. Following upon reduction of pressure to 1.5 bar obtained via a pressure reducer PR, gaseous oxygen is obtained.

An ozone generator or ionizer (OG) comprises a high-voltage generator that ionizes part of the oxygen molecules converting them into ozone (O3). The gas generated by the ozone generator comprises gaseous ozone in a concentration of between 0.06 kg/m3 and 0.14 kg/m³, preferably 0.08 kg/m³.

In one or more embodiments, the ozone-containing gas is introduced directly into the reactor in which the sterilized material is contained, preferably into an autoclave. The ozone-containing gas under pressure (maximum pressure of 1.5 bar) may be injected, by difference of pressure between the ozone generator OG and the inside of the autoclave AC, gradually into the autoclave AC through a steam-inlet ducting.

The inside of the autoclave AC is initially at negative pressure and progressively, by introducing the ozone-containing gas, the pressure increases, reaching a value comprised between 0.3 bar and 1.5 bar, preferably equal to 0.5 bar.

Treatment of the sterilized post-consumer absorbent sanitary products with gaseous ozone may be carried out for a period of time comprised between 30 min and 90 min, preferably equal to 60 min. At the end of the period of time indicated, the gas containing ozone in excess, which has not reacted, is eliminated from the autoclave AC, for example by means of a vacuum pump VP, as schematically illustrated in FIG. 4.

At the end of the oxidizing treatment, the material at output from the autoclave may be collected in a storage container 32, for example illustrated in FIG. 2. After the step of treatment with ozone, the products may be subjected to the shredding step, the drying step, and the separation step, as described in what follows.

From the storage container 32 the sterilized and decontaminated material is sent on to a shredder 36 via a conveyor belt 38. The shredder may comprise, for example, two rotors driven by a motor. The rotors are provided with teeth that carry out shredding of the material. Shredding enables shredded material to be obtained having a particle size smaller than 10 cm, preferably smaller than 3 cm, more preferably smaller than 1 cm. After shredding SH, the material may present a density in the region of 400-500 kg/m³, a temperature of approximately 75-95° C., and a humidity in the region of 70-85%.

The material subjected to the steps of sterilization SR, decontamination DC, and shredding SH is sent on via a conveyor 44 to a drier 42, where the drying step DR is carried out. The drier 42 comprises a casing, housed in which are perforated horizontal conveyors, driven alternately in opposite directions and set on top of one another in a vertical direction. The conveyor 44 unloads the material onto the top conveyor. At output from each horizontal conveyor the material drops onto the underlying conveyor. While the material is conveyed in a horizontal direction and passes in sequence from one conveyor to the underlying one, a flow of heated air traverses the casing from beneath upwards. The flow of air traverses the perforated conveyors and the material located thereon. The flow of air is generated by a fan 50 connected to a filter. The flow of air is heated in a battery of heat exchangers 54 supplied with steam. The flow of air at output from the heat exchanger 42 is sucked in by a second fan and is sent on to a condensation-discharge device and a scrubber. At output from the drier 42, the material is unloaded onto a conveyor belt 62. The drier 42 may be provided with microwave generators facing the top conveyor in order to accelerating heating of the material and thus increase the drying effect. The material at inlet to the drier has a temperature of approximately 70-90° C. The temperature of the drying air within the drier 42 is approximately 140° C. The product at output from the drier 42 has a temperature of approximately 50-70° C., a density of approximately 35-50 kg/m³, and a humidity of between 5% and 20%.

Downstream of the drying step DR, the sterilized, decontaminated, shredded, and dried material is sent on to a separation assembly 64, in which the step of separation of plastic and cellulose (SEP I) is carried out. The separation assembly 64 may comprise at least one first centrifugal separator including a base and having an inlet for the material to be separated. In the example illustrated in FIG. 2, two centrifugal separators 66, 67 are provided arranged in cascaded fashion. The centrifugal separator 66 may comprise a separation chamber housed in which is a perforated cylindrical filter, in which a rotor is mounted rotatably about a horizontal axis. The material at inlet is projected in a radial direction from inside towards the outside against the perforated filter. The cellulose has a smaller particle size than the plastic and passes through the filter and is collected in a first outlet, whereas the plastic remains inside with respect to the filter and is collected in a second outlet. Preferably, the plastic at output from the first centrifugal separator 66 is sent on to a second centrifugal separator 67 having a filter with smaller perforations. At output from the first centrifugal separator there are obtained cellulose with a purity in the region of 85%-95% and plastic with a purity in the region of 60%-80%. At output from the second centrifugal separator there are obtained cellulose with a purity in the region of 85%-95% and plastic with a purity in the region of 85%-97%.

The plastic at output from the separator 66 can be sent on to a plastic shredder 84 and then to an extruder or densifier 86.

With reference to FIG. 2, at output from the centrifugal separators 66 the flows of cellulose 80 can be sent on to a cellulose shredder and a cellulose pelletizer 82. Alternatively, the flows of cellulose can be sent on for a further separation step SEP 11, to a further separator apparatus for separation of cellulose and SAP to obtain cellulose and SAP with a higher degree of purity. In particular, separation of the SAP from the cellulose in the step SEP II may comprise two steps.

The first separation step SEP II may envisage the use of mechanical separators equipped with a perforated fixed shield and a central rotor. The cellulose fibres of larger size remain withheld by the screen, whereas the fraction comprising the SAP passes through the holes, which have a size comprised between 2 mm and 5 mm, and is conveyed through a second mechanical separator to undergo a second separation step. At output from the second mechanical separator, the fraction of SAP may comprise cellulose fibres of small size that have not been withheld by the shield of the mechanical separators.

The second separation step SEP II may use a further separator, such as an air classifier with a cyclone system equipped with a metal wheel that is able to rotate at a rate of approximately 4500 rpm. The material to be separated is inserted in the top part of the classifier and hit by a flow of air at a flowrate of between 10 m³/min and 30 m³/min. The separator comprises two outlets conveyed in which are the fraction of high-purity SAP and the fraction of cellulose fibres, respectively.

In one or more embodiments, the step of decontamination by means of oxidizing treatment with ozone is carried out downstream of the sterilizing step and prior to the step of shredding of the sterilized absorbent sanitary products (FIG. 3).

The method forming the subject of the present disclosure favours sterilization of the post-consumer absorbent sanitary products and also an effective decontamination from organic compounds as abatement of the possible amount of organic residue of a post-metabolic nature also deriving from use of drugs.

The advantage obtained from carrying out the step of treatment with a gas containing ozone directly on the sterilized products consists in that:
at the end of the sterilizing step SR the products at output from the autoclave have a humidity in the region of 60%-75% and a temperature equal to or higher than 60° C.; this enables elimination of the step of heating and humidification of the material to be subjected to the oxidizing treatment with gaseous ozone;
treatment with gaseous ozone can be carried out in the same reactor, preferably the autoclave, in which the step of sterilization SR of the products has been carried out;
the sterilizing step SR acts on the post-consumer sanitary products deriving from differentiated collection preparing them for a more effective contact with the ozone-containing gas.

The present Inventors have moreover noted that the SAP recovered using the method described maintain a significant absorption capacity (AC). In particular, the absorption capacity presents a reduction of less than 4% as compared to the absorption capacity of pristine SAP. The aforesaid absorption capacity is measured by meting out a pre-weighed amount Mi of sample into an excess of deionized water (liquid-to-solid ratio of 100 g/ml). After complete expansion, the mixture is centrifuged at 1350 rpm for 3 min. The swollen material having a mass M2 is weighed. The absorption capacity is calculated by applying the formula: AC=(M2−Mi); Mi (gig), where Mi and M2 are the weights (in grams) of the anhydrous material and the swollen material, respectively. The method described herein hence enables recycling and marketing of the sanitary products treated as recovered raw materials (and not as waste).

### EXAMPLES

The ensuing description regards experimental tests conducted in order to test the effectiveness of the method forming the subject of the present disclosure in obtaining a decontamination from residual chemical compounds of the treated post-consumer absorbent sanitary products. The following results demonstrate that a decontaminating step carried out by treatment with gaseous ozone of the post-consumer sanitary products subjected to sterilization enables a significant abatement of residual organic compounds to be obtained, even at levels higher than 95%.

### Chemical Contamination of Post-Consumer Sanitary Products Subjected to Sterilization

The effectiveness of the oxidative decomposition performed using ozone was assessed according to the abatement of substances appearing in Table 3b of the Legislative Decree No. 62¹¹ of May 15, 2019. Cellulose samples deriving from post-consumer absorbent sanitary products subjected to sterilization carried out in an autoclave at a temperature of 135° C. and at an internal pressure of 3.1 bar, for a period of 20 min were fortified with the aforesaid substances, in particular using a concentration 100 times higher than the limits appearing in the legislative decree itself. The samples (humidity comprised between 60% and 75%) were subjected to treatment with gaseous ozone using different conditions of pressure and different treatment times. Except for Diazepam, purchase of which is limited by ministerial restrictions since it is a narcotic substance.

The analyses conducted on the aqueous extracts of the materials, first fortified and then treated, revealed that—according to the operating conditions—the substances used as contamination indicators may persist in amounts higher than the limits indicated in the aforesaid legislative decree.

Preliminary results showed that the treatment carried out with a gas containing ozone at a concentration of 0.08 kg/m³ and a pressure of 1.5 bar for a total period of 60 min enables greater effectiveness to be obtained in terms of abatement of the indicator substances with which the samples were fortified upstream.

When the oxidizing treatment was carried out for 60 Min at a pressure of 1.5 bar, the abatement was equal to 93% of the total initial contamination. The percentage abatement dropped with the reduction in pressure; in particular, it dropped to 89% moving to isobar values of exposure to gas, but for exposure times shorter than 20 min, and to 87% moving to switching to isochronous exposure values, but at pressures reduced to one third as compared to the most extreme conditions. The increase of pressure to 1.5 bar, albeit causing an increase in effectiveness from a standpoint of decontamination, may, however, lead to reactions of de-crosslinking and depolymerization under load of the SAP and of the cellulose, respectively. With reference to the bleaching effect, this appears correlated with the times of exposure to gaseous ozone and not to the pressure. The optimal bleaching effect is obtained, in fact, by exposing the material to the ozone-containing gas at a pressure of 0.5 bar for a total period of 60 min.

Further experimental tests were repeated on fortified cellulose samples—as for the preliminary test—using the substances already employed for the tests referred to above and indicated in Table 1 below in a concentration 100 times higher than the limit values appearing in the aforesaid legislative decree (Table 3b of the Legislative Decree No. 62, dated May 15, 2019). The samples were subjected to treatment with gaseous ozone for a period of 60 min, at a pressure of 0.5 bar, at a temperature of 70° C., and kept in motion. The results are provided in Table 1 below and demonstrate a total percentage abatement higher than 95%.

TABLE 1

| | | Pre- | Post- | Limit |
|---|---|---|---|---|
| | | trearment | treatment | value |
| N. | Indicator substances | μg/kg_{ds} | μg/kg_{ds} | μg/kg_{ds} |
| 1 | Clavulanic acid* | <10 | <10 | 10 |
| 2 | Amoxicillin | 394 | <10 | 10 |
| 3 | Ampicillin | 352 | <10 | 10 |
| 4 | Cefaclor (as cefalosporin) | 497 | <10 | 10 |
| 5 | Clarithromycin | 431 | <10 | 10 |
| 6 | Ciprofloxacin | 304 | <10 | 10 |
| 7 | Levofloxacin | 371 | <10 | 10 |
| 8 | Azithromycin | 188 | <10 | 10 |
| 9 | Hydrocortisone acetate | 348 | <7 | 7 |
| 10 | Hydrochlorothiazide | 497 | <25 | 25 |
| | (as benzothiadiazine) | | | |
| 11 | Diazepam (as benzodiazepine)* | <30 | <30 | 30 |
| 12 | Ibuprofen | 892 | <45 | 45 |
| 13 | Diclofenac sodium salt | 963 | <45 | 45 |
| 14 | Acetylsalicylic acid | 425 | <5 | 5 |
| 15 | Bicalutamide | 878 | <50 | 50 |
| 16 | Metformin | 1180 | <50 | 50 |
| 17 | Pioglitazone | 479 | <50 | 500 |
| 18 | Clopidogrel | 715 | <100 | 100 |
| 19 | Lansoprazole | 222 | <3 | 3 |
| 20 | Telmisartan | 390 | <15 | 15 |
| 21 | Torasemide* | <15 | <15 | 15 |
| 22 | Lovastatin | 747 | <35 | 35 |
| 23 | Atorvastatin | 318 | <15 | 15 |
| 24 | Clodronic acid | 128 | <1 | 1 |
| | Total abatement percentage | | 95.2% | |

*Fortification with diazepam, torasemide and clavulanic acid was not carried out on the control sample. The values are expressed as weight ratio (mass of substance to mass of sample dried at 105° C. up to constant weight). The release test was carried out in water with a liquid-to-solid ratio equal to L/S = 100 ml/g (recovery of the eluates at 24 h of soaking). Substances extracted at alkaline pH according to law (Legislative Decree No. 62 dated May 15, 2019).

The specific operating conditions of the steps of the method forming the subject of the present disclosure make it possible to favour effective decontamination, obtain an effect of bleaching and elimination of odours, and at the same time counter degradation of the material to he recovered and marketed for re-use.

Of course, without prejudice to the principle of the invention, the details of construction and the embodiments may vary widely, without thereby departing from the scope of the invention, as defined by the ensuing claims.

1. A method for sterilizing and decontaminating post-consumer absorbent sanitary products to be recycled, wherein the post-consumer absorbent sanitary products are contaminated by organic compounds deriving from human metabolism and comprising drug residue, the post-consumer absorbent sanitary products comprising fractions of plastic, super-absorbent polymers (SAP), and optionally cellulose, the method comprising the steps of:
sterilizing (SR) the post-consumer absorbent sanitary products by heating to a temperature equal to or lower than 140° C. and at a pressure comprised between 1 bar and 3.6 bar to obtain sterilized post-consumer absorbent sanitary products; and
decontaminating (DC) from the organic compounds the sterilized post-consumer absorbent sanitary products by means of an oxidizing treatment,
wherein the oxidizing treatment is carried out by putting the sterilized post-consumer absorbent sanitary products in contact with a gas containing ozone,
wherein the oxidizing treatment is carried out at a temperature equal to or higher than 60° C., and
wherein the sterilized post-consumer absorbent sanitary products subjected to the decontaminating step (DC) by means of the oxidizing treatment have a humidity lower than 80%.

2. The method according to claim 1, wherein the post-consumer absorbent sanitary products to be recycled comprise the fractions of plastic, cellulose, and SAP and comprise diapers for babies, incontinence pads for adults, sanitary napkins, bed linings.

3. The method according to claim 1, wherein the concentration of ozone in the gas is comprised between 0.6 kg/m³ and 0.14 kg/m³, and is preferably equal to 0.08 kg/m³.

4. The method according to claim 1, wherein the oxidizing treatment of the decontaminating step (DC) is carried out at a pressure comprised between 0.3 bar and 1.5 bar, preferably equal to 0.5 bar.

5. The method according to claim 1, wherein the oxidizing treatment of the decontaminating step (DC) is carried out for a period of time comprised between 30 min and 90 min, preferably equal to 60 min.

6. The method according to claim 1, wherein the method does not comprise steps of immersion of the post-consumer absorbent sanitary products in water or in aqueous solutions.

7. The method according to claim 1, wherein the sterilizing step (SR) and the subsequent decontaminating step (DC) are carried out in a closed reactor, preferably an autoclave, more preferably a rotary autoclave.

8. The method according to claim 7, wherein the oxidizing treatment of the decontaminating step (DC) is carried out by introducing the ozone-containing gas into said the reactor.

9. The method according to claim 7, wherein the decontaminating step (DC) comprises a step wherein a vacuum condition is created in the reactor, the vacuum-creation step preceding introduction of the ozone-containing gas into the reactor.

10. The method according to claim 1, the method further comprising the step of shredding (SH) the post-consumer absorbent sanitary products sterilized and decontaminated from the organic compounds and obtaining shredded post-consumer absorbent sanitary products having a particle size of less than 10 cm, preferably less than 3 cm, more preferably less than 1 cm.

11. The method according to claim 10, the method further comprising the step of drying (DR) the shredded post-consumer absorbent sanitary products and obtaining shredded and dried post-consumer absorbent sanitary products comprising plastic. SAP, and optionally cellulose.

12. The method according to claim 11, wherein the method further comprises the step of separating plastic, optionally cellulose, and SAP from the shredded and dried post-consumer absorbent sanitary products.

13. The method according to claim 12, wherein the plastic separated from the absorbent sanitary products is decontaminated by organic compounds deriving from human metabolism and comprising drug residue.

14. The method according to claim 12, wherein the cellulose separated from the absorbent sanitary products is decontaminated by organic compounds deriving from human metabolism and comprising drug residue.

15. The method according to claim 12, wherein the SAP separated from the absorbent sanitary products are decontaminated by organic compounds deriving from human metabolism and comprising drug residue. and

16. The method according to claim 15, wherein the absorption capacity (AC) of the super-absorbent polymers separated from the absorbent sanitary products has a reduction of less than 4% as compared to the absorption capacity of pristine super-absorbent polymers.
